(19) United States
(12) Patent Application Publication
(10) Pub. No.: US 2022/0175695 A1
(43) Pub. Date: Jun. 9, 2022

(54) FORTIFICANT COMPOSITION

(71) Applicant: Conopco, Inc., d/b/a UNILEVER, Englewood Cliffs, NJ (US)

(72) Inventors: Girish MURALIDHARAN, Banashankari (IN); Suma MATTATHIL SUKUMARAN, Bangalore (IN); Vetri KUMARAN, Tamil Nadu (IN); Venkataraghavan RAJANARAYANA, Bangalore (IN); Rajeesh Kumar RAMACHANDRAN, Hosur (IN); Pooja TIWARI, Bangalore (IN)

(73) Assignee: Conopco, Inc., d/b/a UNILEVER, Englewood Cliffs, NJ (US)

(21) Appl. No.: 17/607,107

(22) PCT Filed: Apr. 16, 2020
(86) PCT No.: PCT/EP2020/060676

§ 371 Date: Oct. 28, 2021

(30) Foreign Application Priority Data
May 1, 2019 (EP) 19172082.0

(51) Int. Cl. A61K 31/07; A61K 47/26; A61K 47/10; A61K 9/50

(52) U.S. Cl. CPC A61K 31/07; A61K 9/5084; A61K 47/10; A61K 47/26

(57) ABSTRACT

The present invention provides a fortificant composition comprising a sugar derived humectant in the range of 30 to 60 wt %; a solubilizer in the range of 1 to 40 wt %; an encapsulated fat soluble fortificant in the range of 0.001 to 10 wt %; a water soluble fortificant in the range of 0.001 to 10 wt %; and water in the range of 1 to 30 wt %.

### FIELD OF THE INVENTION

The present invention relates to a fortificant composition and a process of manufacture thereof. The invention more particularly relates to a fortificant composition that can be used in a water dispensing device to enable fortification of water.

The invention has been developed primarily for use in drinking water application and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

Having a balanced diet to get requisite vitamins and minerals from daily diet is preferable over getting these from supplements. The required daily intake of vitamins and minerals is defined in terms of Recommended Dietary Allowance (RDA). When people do not eat or get a healthy diet every day, taking a nutrient supplement that contains the recommended levels of vitamins and minerals becomes necessary. Taking dietary supplement is particularly useful for people who have been diagnosed of vitamin or mineral deficiency.

In human beings, water constitutes approximately 70 percent of the body weight and is a crucial constituent of daily diet. On an average, an adult consumes about 1 to 3 litres of water per day driven by thirst. Thus, providing vitamins and minerals through water as a vehicle for fortification is beneficial.

There have been several attempts to provide water compositions fortified with vitamins and minerals while keeping in mind the need for making it free of objectionable colour, odour and taste.

However, when nutrient fortificants to be added as fortificants are both water-soluble and oil-soluble, then it is difficult to stabilize the fortificants in one phase. A method that is adopted in such a case is to use emulsions, which result in a two-phase system, and also since the fortificants are at very low concentration, this leads to a very unbalanced emulsion phase ratio, which is difficult to stabilize.

Therefore, there is a need to have a fortificant composition capable of incorporating both water and oil soluble fortificant components and have physical, chemical and microbiological stability.

### SUMMARY OF THE INVENTION

The present invention provides a fortificant composition capable of incorporating both water and oil soluble fortificant components and have physical, chemical and microbiological stability.

First aspect of the present invention provides a fortificant composition comprising:
a. a sugar derived humectant in the range of 30 to 60 wt %
b. a solubilizer in the range of 1 to 40 wt %
c. an encapsulated fat soluble fortificant in the range of 0.001 to 10 wt %;
d. a water soluble fortificant in the range of 0.001 to 10 wt %;
e. water in the range of 1 to 30 wt %

Second aspect of the present invention provides use of composition according to the first aspect for delivering fat-soluble and water-soluble vitamins in a single composition.

Third aspect of the present invention provides use of composition according to the first aspect for in-line fortification of drinking water in a water dispensing device.

Fourth aspect of the present invention provides a method of making composition according to the first aspect, the method comprising:
a. adding a sugar derived humectant in the range of 30 to 60 wt % and a solubilizer in the range of 1 to 40 wt % to water so as to obtain an aqueous mixture;
b. mixing the encapsulated fat soluble and the water-soluble fortificants to small aliquots of water to form a mixture and stored at a temperature less than 15° C. to obtain a homogenized fortificant aqueous solution;
c. mixing the aqueous mixture from step a and the homogenized fortificant aqueous solution from step b together to obtain the fortificant composition of the first aspect;

wherein step a and step b are independent of each other.

It should be understood that the present invention is not limited in its application to the details of construction and the arrangement of components set forth in the following description or illustrated in the following drawings. The invention is capable of other embodiments and of being implemented in various ways.

The terms “including”, “comprising”, “containing” or “having” and variations thereof as used herein are meant to encompass the items listed thereafter as well as additional items. Unless specified or limited otherwise, the terms “mounted,” “connected,” “supported,” and “coupled” and variations thereof are used broadly and encompass direct and indirect mountings, connections, supports, and couplings.

The description below is intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or the conditions of reaction, physical properties of materials and/or use are to be understood as modified by the word “about”.

Numerical ranges expressed in the format “from x to y” are understood to include x and y. When for a specific feature multiple preferred ranges are described in the format “from x to y”, it is understood that all ranges combining the different endpoints are also contemplated.

### DETAILED DESCRIPTION OF THE INVENTION

It should be understood that the present invention is not limited in its application and is capable of other embodiments and of being implemented in various ways.

The present invention provides a fortificant composition capable of incorporating both water and oil soluble fortificant components and have physical, chemical and microbiological stability. The fortificant composition is particularly suited for composition use in a water dispensing device to enable fortification of water.

When the present inventors were looking for a solution to fortify water and especially in an in-line system with a water dispensing device by a liquid fortificant composition, they realised that it is difficult to stabilize both water soluble and fat soluble such as water soluble and fat soluble vitamins in one phase. It was also required that the liquid formulation needs to have physical, chemical and microbiological stability, in open shelf and closed shelf conditions, over period of time such as 6 to 18 months, while achieving Constant water activity, minimal degradation of vitamins, constant dosing of micronutrients and keeping the inherent appearance and taste of the fortified water to the consumers appeal.

The present inventors surprisingly found that the fortificant composition of the present invention lead to a more stable, single phase fortificant composition being able to incorporate all kinds of nutrient fortificants irrespective of their solubility in water or oil medium.

Mineral Water Composition

The present invention provides a fortificant composition comprising:
a. a sugar derived humectant in the range of 30 to 60 wt %
b. a solubilizer in the range of 1 to 40 wt %
c. an encapsulated fat soluble fortificant in the range of 0.001 to 10 wt %;
d. a water soluble fortificant in the range of 0.001 to 10 wt %;
e. water in the range of 1 to 30 wt %

It is preferable that the sugar derived humectant is a food grade humectant and more preferably selected from the group of sugar alcohols, monosaccharides and disaccharides.

It is preferable that the solubilizer is a solubility excipient selected from the group of polymer, surfactant and lipid based excipient.

It is preferable that the fat soluble fortificant is a micronutrient and more preferably a fat soluble fortificant and more preferably a fat soluble vitamin.

It is preferable that the encapsulated fat soluble nutrient fortificant is water dispersible.

It is preferable that the water soluble fortificant is preferably a water soluble micronutrient and more preferably a water soluble vitamin.

The present invention provides use of the composition of first aspect for delivering fat-soluble and water-soluble vitamins in a single composition.

The present invention provides use of the composition of first aspect for in-line fortification of drinking water in a water dispensing device.

Sugar Derived Humectant Humectant is a hygroscopic substance which itself absorbs or helps another substance to absorb and retain moisture. A sugar derived humectant has sugar or saccharide as main component. The sugar derived humectant is present in the range of 30 to 60 wt % by weight in the present composition, more preferably in the range of 35 to 58 wt % and most preferably in the range of 40 to 55 wt %.

It is preferable that the sugar derived humectant is a food grade humectant. It is further preferable that the sugar derived humectant is selected from the group of sugar alcohols, monosaccharides and disaccharides.

The preferred sugars for the sugar derived humectant are sugar polyols, sorbitol, xylitol, maltitol, mannitol, lactitol, glycerol, erythritol and the likes and mixtures or combinations thereof. It is more preferable that the preferred sugar is sorbitol or glycerol or mixture or combination thereof. The most preferable sugar is sorbitol. It is preferred that sorbitol is present in the range of 30 to 60% by weight in the present composition, more preferably in the range of 35 to 58 wt % and most preferably in the range of 40 to 55 wt %.

It is preferred that sugar derived humectant and solubilizer are not the same compound. The wt % value of the sugar derived humectant will not alter the required amount of 1 to 40 wt % of solubilizer based on the weight of the composition. It is preferred that the amount of sugar derived humectant should be considered as independently of the amount of solubilizer.

Solubilizer

Solubilizer is a substance which helps in solubilizing the ingredients, acting as a solvent. It is preferred that the solubilizer is a solubility excipient selected from the group of polymer, surfactant, lipid based excipient combinations and mixtures thereof. It is highly preferable that the solubilizer of the present invention is a non-foaming type of solubilizer.

The solubilizers of the present invention comprise 1 to 40 wt %, preferably 5 to 30 wt % and more preferably 10 to 25 wt % by weight of the present composition.

When solubilizer comprises a polymer, the polymer is selected from the group of polymers which can either swell in water or can complex with the actives, such as cross povidone, polyethylene glycol, propylene glycol, mixtures and combinations thereof and the likes.

When solubilizer comprises a surfactant, it is selected from the group of non-ionic surfactants such as polysorbate and the likes.

When solubilizer comprises a lipid based excipient, it is selected from the group of lipophilic surfactants such as sodium lauryl sulfate and the likes

It is preferable that the solubilizer is an emulsifier which will stabilise them and will prevent any phase separation.

It is preferable that solubilizers are selected from the group of polyethylene glycol, polysorbate, hydroxypropyl cellulose, cyclodextrin, starch, sodium lauryl sulfate, mesoporous silica, crosspovidone and propylene glycol, of which propylene glycol is the most preferable of all.

It is most preferable that propylene glycol is used as a solubilizer for the present invention. It is a synthetic food additive having many beneficial properties such as anti-caking, texturizing, emulsifiers, humectant and antioxidant. It is preferable that propylene glycol comprises 1 to 40 wt %, preferably 5 to 30 wt % and more preferably 10 to 20 wt %.

It is preferred that the solubilizer and the sugar derived humectant are not the same compound. The wt % value of the solubilizer will not alter the required amount of 30 to 60 wt % sugar derived humectant based on the weight of the composition. It is preferred that the amount of solubilizer should be considered as independently of the amount of solubilizer.

Encapsulated Fat Soluble Fortificant

A fat soluble fortificant of the present invention is preferably a micronutrient that solubilizes in fat, preferably one or more fat soluble vitamins. Fat-soluble nutrients require the presence of fat to be properly absorbed. Fat soluble nutrients include vitamins A, D, E and K.

It is preferred that the encapsulated fat soluble fortificant is encapsulated in a water dispersible capsule and more preferably a capsule which is dispersible even in cold water.

Encapsulation is a process through which a protective coating is applied to an active. This is very well established technological process in food and pharma industries to either mask the taste or for controlled release of actives. One such example is the Vitamin A palmitate oil distributed in microdroplets of starch derivative matrix. Starch coating enables water dispersibility of fat-soluble fortificant. Any other matrix similar to starch can also be used.

An encapsulated fat soluble fortificant of the present invention is present in the range of 0.001 to 10 wt %, more preferably in the range of 0.01 to 8 wt % and most preferably in the range of 0.01 to 5 wt % of the total weight of the composition.

Vitamin A plays an important role in maintaining healthy vision and immune system. It consists of group of compounds known as retinoids. They can be obtained from plant and animal sources such as fish liver oil, liver of animals, butter. The proactive form such as carotenoids can be obtained from spinach, carrot, kale.

Vitamin D is also group of compounds commonly known as calciferol and broadly classified as Vitamin D2 (plant source) and Vitamin D3 (animal source). Once vitamin D is absorbed into the bloodstream, the liver and kidneys change calciferol into calcitriol, the biologically active form of vitamin D. Vitamin D plays important roles in bone maintenance and supports immune system. Its major sources include fish oil, fatty fish, mushrooms exposed to sunlight and fortified dairy products.

Vitamin E is an antioxidant which helps in destroying free radicals in fatty tissues which could lead to cancer. Tocopherol is most abundant type of vitamin E. Vitamin C and B helps in aiding its functions. Its best sources include wheat germ oil, sunflower oil, hazelnuts, almonds.

Vitamin K has crucial roles in blood clotting and prevents excessive bleeding once the person gets wound. It has two types Vitamin K-1 (Plant sources) and Vitamin K-2 (animal sources). vitamin K can also help with reducing risk of heart disease, bone health, reducing the build-up of calcium in the blood. Common sources include kale, liver, spinach, butter, parsley, egg yolk.

Water Soluble Fortificant

A water soluble fortificant of the present invention is preferably a micronutrient that solubilizes in water, more preferably one or more water soluble vitamins. Water-soluble fortificants require the presence of water to be properly absorbed. Water soluble micronutrients include vitamins. vitamin B and C dissolve in water in the small intestine and are then carried off to the body through the bloodstream.

The water soluble fortificant of the present invention is present in the range of 0.001 to 10 wt %, more preferably in the range of 0.01 to 8 wt % and most preferably in the range of 0.01 to 5 wt % of the total weight of the composition.

Nine water-soluble vitamins are found in the human diet are generally Vitamin B and C and more particularly Vitamin B1 (thiamine), Vitamin B2 (riboflavin), Vitamin B3 (niacin), Vitamin B5 (pantothenic acid), Vitamin B6, Vitamin B7 (biotin), Vitamin B9, Vitamin B12 (cobalamin), Vitamin C. Each vitamin B has their own specific function but commonly vitamin B either act as coenzymes or helps in formation of coenzymes such as vitamin B5 helps in formation of coenzyme A, which is necessary for the synthesis of fatty acids, amino acids, steroid hormones, neurotransmitters and various other important compounds. Coenzymes are small compounds that help enzymes trigger chemical reactions that otherwise wouldn't happen on their own. Vitamin B is found in virtually all animal-sourced foods but is absent from plant foods.

Vitamin C is one of the body's main antioxidants and is required for collagen synthesis. Immune cells contain high levels of vitamin C. Unlike the B vitamins, Vitamin C doesn't act as a coenzyme, although it is a cofactor for prolyl hydroxylase, an enzyme that serves an essential role in the formation of collagen. The main dietary sources of vitamin C are fruits and vegetables.

Water

The fortificant composition of the present invention is a liquid composition and comprises water in the range of 1 to 35 wt %, more preferably 5 to 30 wt % and most preferably 10 to 28 wt % of the weight of present composition

It is preferable that pH of water is in the range of 6.0 to 8.5 and more preferably in the range of 6.5 to 7.5.

Method

The present invention provides a method of preparing fortificant composition of the first aspect, the method comprising:
a. adding a sugar derived humectant in the range of 30 to 60 wt % and a solubilizer in the range of 1 to 40 wt % to water so as to obtain an aqueous mixture;
b. mixing the encapsulated fat soluble and the water-soluble fortificants to small aliquots of water to form a mixture and stored at a temperature less than 15° C. to obtain a homogenized fortificant aqueous solution;
c. mixing the aqueous mixture from step a and the homogenized fortificant aqueous solution from step b together to obtain the fortificant composition of the first aspect;

wherein step a and step b are independent of each other.

It is preferable that in the method of the present invention the mixture of step b is stored at a temperature less than 15° C. for a maximum of 60 to 90 mins and more preferably less than 60 minutes and further preferably less than 30 minutes and most preferably less than 15 minutes.

It is preferable that in the method of the present invention the sugar derived humectant is a food grade humectant and more preferably selected from the group of sugar alcohols, monosaccharides and disaccharides.

It is preferable that in the method of the present invention the solubilizer is a solubility excipient selected from the group of polymer, surfactant and lipid based excipient.

It is preferable that in the method of the present invention the fat soluble fortificant is a nutrient fortificant and more preferably a fat soluble vitamin.

It is preferable that in the method of the present invention the encapsulated fat soluble nutrient fortificant is water dispersible.

It is preferable that in the method of the present invention the water soluble nutrient fortificant is a water soluble vitamin.

The present invention provides use of the composition of first aspect for delivering fat-soluble and water-soluble vitamins in a single composition.

The present invention provides use of the composition of first aspect for in-line fortification of drinking water in a water dispensing device.

### EXAMPLES

Technical benefits of the disclosed invention will now be explained further with the help of a non-limiting example.

### Example 1

Fortificant Composition

The preferred fortificant composition (P1) of the present invention was prepared by taking 50 g of sorbitol and adding 20 g of propylene glycol to obtain a mixture which was kept for stirring at 90-120 rpm using overhead stirrer. About 0.5 g of deionized water was taken in a container and 0.1 g of sodium benzoate (a preservative) was dissolved and add to the stirring mixture. About 0.25 g of encapsulated fat soluble vitamins and 0.02 g of water soluble vitamins were dissolved and the mixture was kept at a low temperature of (4° C.) for 5-10 minutes. 5 g of Sorbitol was then added to the above cooled mixture, stirred manually and was added to the e stirring mixture kept at overhead stirrer to obtain the fortificant composition as provided in Table 1 as P1. Similarly P2 was prepared using PEG30 & Tween 20 as solubilizers and P3 was prepared without solubilizers

TABLE 1
Composition of Fortificant

| Ingredients | P1 (w/w) % | P2 (w/w) % | P3 (w/w) % |
|---|---|---|---|
| Fat soluble vitamin(s) | 0.25 | 0.3 | 0.26 |
| Water soluble vitamin(s) | 0.02 | 0.02 | 0.008 |
| Solubilizer (Propylene glycol) | 20.00 | — | — |
| Solubilizer (PEG30 & Tween 20) | — | 23 | — |
| Sorbitol (Sugar based | 55 | 55 | 68.4 |
| humectant) | | | |
| Water and Minors | Made upto | Made upto | Made upto |
| | 100 | 100 | 100 |

### Example 2

Water Activity of the Fortificant Composition

Fortificant having composition P2 and P3 mentioned in Table 1 was tested for its water activity in AquaLab Series 3TE water activity meter. 5 ml of fortificant was transferred to the sample cup and placed inside the activity meter. The water activity and temperature readings were noted down. The readings represent, an average of three water activity readings.

Table 2 and 3 correspond to the water activity measurements of P2 taken at room temperature (RT) and room humidity (RH) (50-60%) and at high humidity (HH) (85-90%) respectively. For room humidity measurements, the fortificant was kept in pin holed lid container and for high humidity the fortificant was kept in pin holed lid container in Rinac India humidity chamber having DRI-FOG humidifier (V 3.1).

TABLE 2
RH and RT

| | Fortificant Water |
|---|---|
| Number of Days | Activity |
| 0 | 0.68 |
| 1 | 0.67 |
| 3 | 0.68 |
| 15 | 0.68 |
| 16 | 0.68 |
| 42 | 0.68 |

TABLE 3

| | Fortificant Water |
|---|---|
| Number of Days | Activity |
| 1 | 0.67 |
| 3 | 0.68 |
| 15 | 0.68 |
| 25 | 0.68 |
| 40 | 0.69 |

From the tables 2 & 3, it can be inferred that water activity of the formulation remains constant at both RH and HH conditions. Water activity is indirect measure of shelf life.

Similarly, Table 4 corresponds to the water activity measurements taken for the fortificant having composition P3 starting at ambient condition and then moving to high humidity (HH) (85-90%) conditions. The measurements were taken as described above.

TABLE 4

| Number of Days | Fortificant Water Activity |
|---|---|
| Day 0 | 0.68 at ambient condition |
| Day 1 | 0.69 |
| Day 3 | 0.68 |
| Day 5 | 0.70 |
| Day 10 | 0.78 |

A water activity of below 0.70 indicates good shelf life, whereas water activity above 0.7 indicates an unstable composition.

It can be concluded from data presented in the tables above that in absence of the solubilizers the P3 fortificant composition is not stable as P1 and P2 compositions for which the water activity remains constant and below 0.70 even at HH for the data taken for about 40 days. On the other had the composition of P3 is unstable at 10^{th} day itself in HH condition.

### Example 3

Vitamin a Stability

Fortificant P2 mentioned in Table 1 was analysed for its Vitamin A stability in HPLC.

Sampling procedure: About 23.5 g Methanol was taken in a container and 1 g of fortificant was added to it. Thereafter 2 ml of Dimethyl Sulfoxide (DMSO) was added followed up by adding 74.5 g water. Theoretically, 1 g of fortificant taken corresponds to 3.2 ppm of Vitamin A. The results of the test are as provided in table 4.

HPLC analysis conditions: Mobile phase consists of Methanol:Ethyl Acetate (70:30), flow rate is set at 1 ml/min, injection volume as 20 μL and analysis time as 20 minutes. Detector used was PDA.

TABLE 4

| Days Interval | Measured Vit A (ppm) |
|---|---|
| 0 | 3.2 |
| 7 | 3.1 |
| 8 | 3.0 |
| 11 | 2.7 |
| 22 | 2.2 |

From table 4, it can be seen that Vitamin A concentration has not degraded below 70% (2.2 ppm) over 3 weeks from the initial value of 3.2 ppm. Vitamin A in the presence of water otherwise would not be stable even for 4 hours.

### Example 4

Microbiological Stability

Fortificants P1 and P2 mentioned in Table 1 were checked on Day 1 for microbial contamination. Media used were TSA (for bacteria) and PDA (for fungus). Spread plate method was used for fungus with 0.3 ml of fortificant and pour plate methods was used for bacteria with 1 ml of fortificant respectively. Plating was done for undiluted and −2 dilution of samples serial diluted in saline.

Bacteria plated were incubated at 37° C. for 24 hrs and growth were observed if any. PDA plated were incubated for 72 hrs at 30° C. to check the contamination.

Fortificant sample testing was repeated to check any microbial growth after 3 weeks while keeping the sample at room temperature.

The results of the test are as provided in table 5.

TABLE 5

| | Count/ml | |
|---|---|---|
| Agar (Medium) | Immediate | After 3 weeks |
| TSA (Bacteria) | 0 | 0 |
| PDA (Fungus) | 0 | 0 |

It can be inferred that there is no growth in terms of bacteria and fungus over a period of time.

### Example 5

Dosing

Fortificants P1 and P2 mentioned in Table 1 were tested for dosing of the fortificants.

50 ml of fortificant having viscosity 180 cP and P1/P2 was taken in sample container and connected to a dosing pump with the flow rate 1 ml/min and operating voltage 4.5V. The dosing pump is procured from Kamoer, and the model number is KPP-DE-S01W.

The pump was consistently dosing 0.5 ml/L fortificant. Different volumes of fortified water were taken and checked for conductivity measurements.

The conductivity of fortified water tested at different volumes of water, while being dosed from the device is provided in table 6.

TABLE 6

| Volume | Conductivity (μS/cm) |
|---|---|
| (mL) of fortified water tested | (n = 4) |
| 100 | 4.4 |
| 1000 | 4.5 |
| 2000 | 4.6 |

The results indicate that a constant dose is delivered across different volumes of water drawn, and is equal to desired 7.5% RDA/L

The various features of the present invention referred to in individual sections above apply, as appropriate, to other sections mutatis mutandis. Consequently, features specified in one section may be combined with features specified in other sections as appropriate. Any section headings are added for convenience only and are not intended to limit the disclosure in any way.

1-13. (canceled)

14. A fortificant composition comprising:
a. a sugar derived humectant, selected from the group of sugar alcohols, monosaccharides and disaccharides, in the range of 30 to 60 wt %;
b. a solubilizer in the range of 5 to 30 wt %; wherein the solubilizer is a solubility excipient selected from the group of polymer, surfactant and lipid based excipient; an encapsulated fat-soluble micronutrient in the range of 0.001 to 10 wt %;
c. a water-soluble micronutrient in the range of 0.001 to 10 wt %; and
d. water in the range of 1 to 30 wt %;
wherein the sugar derived humectant is a sugar polyol, the solubilizer is cross povidone, polyethylene glycol, propylene glycol or mixtures thereof as a polymer; the solubilizer is polysorbate as a surfactant; and the water-soluble and encapsulated fat-soluble micronutrients are vitamins.

15. The composition according to claim 14 wherein the sugar derived humectant is a food grade humectant.

16. The composition according to claim 14 wherein the encapsulated fat-soluble micronutrient is water dispersible.

17. The composition according to claim 14 wherein the composition is suitable to deliver fat-soluble and water-soluble vitamins in a single composition.

18. The composition according to claim 14 wherein the composition suitable for in-line fortification of drinking water in a water dispensing device.

19. A method for making the composition according to claim 1, the method comprising the steps of:
a. adding a sugar derived humectant in a range of 30 to 60 wt % and a solubilizer in a range of 5 to 30 wt % to water to obtain an aqueous mixture;
b. mixing the encapsulated fat-soluble and the water-soluble micronutrient to aliquots of water to form a mixture and storing the mixture at a temperature less than 15° C. to obtain a homogenized fortificant aqueous solution;
c. mixing the aqueous mixture from step a and the homogenized fortificant aqueous solution from step b together to obtain the fortificant composition;
wherein step a and step b are independent of each other.

20. The method according to claim 19 wherein the mixture of step b is stored at a temperature less than 15° C. for a maximum of 60 to 90 minutes.
